SEQUENCE LISTING (2 sequences)

SEQ ID NO: 1 - DNA, 25 bp, Artificial
  Synthetic
ctcccagcgagtacagccccgccat

SEQ ID NO: 2 - DNA, 25 bp, Artificial
  Synthetic
cggtcccacgcagcccgccgaatcc

(19) United States
(12) Patent Application Publication
(10) Pub. No.: US 2004/0167189 A1
(43) Pub. Date: Aug. 26, 2004

(54) Materials and methods for inhibiting Wip1

(75) Inventors: Dmitry Bulavin, Washington, DC (US); Galina Belova, Rockville, MD (US); Albert J. Fornace, Bethesda, MD (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: The Government of the U.S.A., as Represented by the Secretary, Dept. of Health and Human Services, Rockville, MD (US)

(21) Appl. No.: 10/800,296

(22) Filed: Mar. 12, 2004

Related U.S. Application Data
(63) Continuation-in-part of application No. PCT/US03/08997, filed on Mar. 21, 2003.
(60) Provisional application No. 60/366,883, filed on Mar. 22, 2002.

(51) Int. Cl. A61K 31/4245; A61K 31/185

(52) U.S. Cl. 514/364; 514/553

(57) ABSTRACT

The invention provides a method of treating cancer in a mammal that expresses the same level or a higher level of Wip1 as compared to a mammal of the same species that does not have cancer, and a method of inhibiting Wip1 in a cell. The invention also provides pharmaceutical compositions comprising a compound selected from the group consisting of Compounds A-N, and a method of making a cancer therapeutic composition. Also provided is a compound selected from the group consisting of Compounds A-N, a method of screening a compound for Wip1-inhibiting activity, and a method of determining the efficacy with which a test compound inhibits Wip1.

### CROSS-REFERENCE TO RELATED PATENT APPLICATIONS

This patent application claims the benefit of International Patent Application No. PCT/US03/08997, filed Mar. 21, 2003, and U.S. Provisional Patent Application Ser. No. 60/366,883, filed Mar. 22, 2002.

### FIELD OF THE INVENTION

This invention pertains to a method of treating cancer in a mammal, a method of inhibiting Wip1 in a cell, a pharmaceutical composition, compounds thereof, and a method of making a cancer therapeutic composition. The present invention further pertains to a method of screening a compound for inhibiting Wip1 activity, in addition to a method of determining the efficacy with which a test compound inhibits Wip1 phosphatase activity.

### BACKGROUND OF THE INVENTION

Wild-type p53-induced phosphatase 1 (Wip1) is a Mg²⁺-dependent serine/threonine protein phosphatase that is expressed in response to ionizing or ultra-violet (UV) radiation in a manner that is dependent on the tumor suppressor gene product p53. Its role in cancer was first suggested by Fiscella et al., Proceedings of the National Academy of Sciences, U.S.A. 94: 6048-6053 (1997), which reported Wip1 as an important inhibitor of growth, since ectopic expression of WIP1 (also known as PPMD1) in a human glioblastoma cell line (T98G) resulted in fewer colonies of cells. In contrast to these results, Wip1 was shown by Takekawa et al., EMBO Journal 19(23): 6517-6526 (2000), to dephosphorylate the kinase p38, which functions to activate p53 for the induction of apoptosis and transcription in response to environmental stress, thereby rendering Wip1 anti-apoptotic as opposed to anti-proliferative. Further studies have confirmed the results of Takekawa et al., and have demonstrated a potential role for Wip1 as a candidate proto-oncogene involved in tumorigenesis (see, e.g., Bulavin et al., Nature Genetics, 31(2), 210-215 (2002)).

In view of the foregoing, the present invention provides materials and methods for treating cancer in a mammal that expresses elevated levels of Wip1. These and other advantages of the invention, as well as additional inventive features, will be apparent from the description of the invention provided herein.

### BRIEF SUMMARY OF THE INVENTION

The present invention provides an isolated or purified oligonucleotide consisting essentially of the nucleotide sequence of SEQ ID NO: 1 or SEQ ID NO: 2, as well as an isolated or purified morpholino oligomer consisting essentially of the nucleotide sequence of SEQ ID NO: 1 or SEQ ID NO: 2.

The present invention further provides a method of detecting cancer or a predisposition to cancer in a mammal. The method comprises comparing the level of expression of Wip1 in a test sample comprising Wip1 obtained from the mammal to the level of expression of Wip1 in a control sample. A higher level of expression of Wip1 in the test sample as compared to the control sample is indicative of cancer or a predisposition to cancer in the mammal.

Further provided by the present invention is a method of treating cancer in a mammal that expresses the same level or a higher level of Wip1 as compared to a mammal of the same species that does not have cancer. The method comprises administering to the mammal a cancer-treating effective amount of a Wip1 inhibitor.

The present invention also provides a method of inhibiting Wip1 phosphatase activity in a cell. The method comprises administering to the cell a compound selected from the group consisting of Compound A, Compound B, Compound C, Compound D, Compound E, Compound F, Compound G, Compound H, Compound I, Compound J, Compound K, Compound L, Compound M, and Compound N, or a pharmaceutically-acceptable salt of any of the foregoing, in an amount effective for inhibiting Wip1 in a cell.

The present invention also provides a method of screening an oligonucleotide or morpholino oligomer for the ability to inhibit the expression of Wip1. The method comprises comparing the level of expression of Wip1 in a test sample obtained from Wip1-expressing cells that have been contacted with the oligonucleotide or morpholino oligomer to the level of expression of Wip1 in a control sample obtained from Wip1-expressing cells that have not been contacted with the oligonucleotide or morpholino oligomer. A lower level of expression of Wip1 in the test sample as compared to the control sample is indicative of the ability of the oligonucleotide or morpholino oligomer to inhibit the expression of Wip1.

A method of determining the efficacy with which a test oligonucleotide or morpholino oligomer inhibits Wip1 expression is further provided by the present invention. The method comprises comparing the level of expression of Wip1 in a test sample obtained from Wip1-expressing cells that have been contacted with the test oligonucleotide or morpholino oligomer to the level of expression of Wip1 in a control sample obtained from Wip1-expressing cells that have been contacted with an oligonucleotide or morpholino oligomer that is known to inhibit the expression of Wip1. A lower level of expression of Wip1 in the test sample as compared to the control sample is indicative of the test oligonucleotide or morpholino oligomer having a greater efficacy for inhibiting the expression of Wip1 than the known oligonucleotide or morpholino oligomer, whereas an higher level of expression of Wip1 in the test sample as compared to the control sample is indicative of the test oligonucleotide or morpholino oligomer having a lower efficacy for inhibiting the expression of Wip1 than the known oligonucleotide or morpholino oligomer.

Further provided is a method of screening a compound for Wip1-inhibiting activity. The method comprises comparing the level of Wip1 phosphatase activity in a test sample that has been contacted with the compound to the level of Wip1 phosphatase activity in a control sample that has not been contacted with the compound. A lower level of Wip1 phosphatase activity in the test sample as compared to the control sample is indicative of the ability of the compound to inhibit Wip1.

The present invention also provides a method of determining the efficacy with which a test compound inhibits Wip1. The method comprises comparing the level of Wip1 phosphatase activity in a test sample that has been contacted with the test compound to the level of Wip1 phosphatase activity in a control sample that has been contacted with a compound that is known to inhibit Wip1. A lower level of Wip1 phosphatase activity in the test sample as compared to the control sample is indicative of the test compound having a greater efficacy for inhibiting Wip1 than the known compound, whereas a higher level of Wip1 phosphatase activity in the test sample as compared to the control sample is indicative of the test compound having a lower efficacy for inhibiting Wip1 than the known compound.

The present invention also provides a compound selected from the group consisting of Compound A, Compound B, Compound C, Compound D, Compound E, Compound F, Compound G, Compound H, Compound I, Compound J, Compound K, Compound L, Compound M, and Compound N, or a pharmaceutically-acceptable salt of any of the foregoing. Pharmaceutical compositions comprising a compound selected from the above group, or a pharmaceutically-acceptable salt of any of the above compounds, and a pharmaceutically-acceptable carrier are also provided.

A method of making a cancer therapeutic composition is further provided by the present invention. The method comprises formulating a compound selected from the group consisting of Compound A, Compound B, Compound C, Compound D, Compound E, Compound F, Compound G, Compound H, Compound I, Compound J, Compound K, Compound L, Compound M, and Compound N, or a pharmaceutically-acceptable salt of any of the foregoing, with a pharmaceutically-acceptable carrier.

### DETAILED DESCRIPTION OF THE INVENTION

The present invention provides isolated or purified oligonucleotides comprising, consisting essentially of, or consisting of, the nucleotide sequence of SEQ ID NO: 1 or SEQ ID NO: 2, which can be used in the methods described herein. The term “isolated” as used herein means having been removed from its natural environment. The term “purified” as used herein means having been increased in purity, wherein “purity” is a relative term, and not to be construed as absolute purity. The term “oligonucleotide” as used herein means a polymer of DNA or RNA (i.e., a polynucleotide), which can be single-stranded or double-stranded, which can be synthesized or obtained from natural sources, and which can contain natural, non-natural, or altered nucleotides. With respect to the isolated or purified oligonucleotides of the present invention, it is preferred that no insertions, deletions, inversions, and/or substitutions are present in the oligonucleotide. However, it may be suitable in some instances for the isolated or purified oligonucleotide of the present invention to comprise one or more insertions, deletions, inversions, and/or substitutions. It is, furthermore, preferred that the isolated or purified oligonucleotides of the present invention are synthesized, single-stranded polymers of DNA.

Isolated or purified morpholino oligomers comprising, consisting essentially of, or consisting of, the sequence of SEQ ID NO: 1 or SEQ ID NO: 2 are further provided by the present invention. The term “morpholino oligomer” as used herein refers to a polymer of the genetic nitrogeneous bases, adenine, guanine, cytosine, and thymine, in which the nitrogenous bases are linked to a 6-membered morpholine ring, as opposed to ribose or deoxyribose as in RNA or DNA. The term “genetic nitrogenous base” as used herein refers to the four nitrogenous bases of deoxyribonucleic acid (DNA) (i.e., adenine, guanine, cytosine, and thymine). Also, each unit of the morpholino oligomer that comprises the nitrogeneous base and the morpholine ring is bridged to neighboring units via phosphorodiamidate linkages, in contrast to the phosphodiester linkages of oligonucleotides. For more description of morpholino oligomers, see Summerton et al., Antisense & Nucleic Acid Drug Development 7:187-195 (1997).

The sequence of the nitrogeneous bases of the morpholino oligomers is essential for their use in the methods described herein. Like the oligonucleotides of the present invention, it is preferred that the morpholino oligomers do not comprise any insertions, deletions, inversions, and/or substitutions. However, it may be suitable in some instances for one or more insertions, deletions, inversions, and/or substitutions to be present in the morpholino oligomers of the present invention. It is, furthermore, preferred that the isolated or purified morpholino oligomers of the present invention are single-stranded.

A variety of techniques used to synthesize the oligonucleotides of the present invention are known in the art. See, for example, Lemaitre et al., Proceedings of the National Academy of the Sciences 84: 648-652 (1987), and Fiscella et al, supra. Likewise, a variety of techniques employed for the synthesis of morpholino oligomers are known in the art. See, for example, U.S. Pat. No. 5,185,444. Alternatively, oligonucleotides or morpholino oligomers can be purchased from companies, such as Eurogentec, Belgium (for oligonucleotides) and Gene Tools, Philomath, Oreg. (for morpholino oligomers).

The present invention further provides a method of detecting cancer or a predisposition to cancer in a mammal. The method comprises comparing the level of expression of Wip1 in a test sample comprising Wip1 obtained from the mammal to the level of expression of Wip1 in a control sample, wherein the control sample is a sample comprising Wip1 taken from a mammal, desirably of the same species, which is known to not have cancer or a predisposition to cancer. In this method, a higher level of expression of Wip1 in the test sample as compared to the control sample is indicative of cancer or a predisposition to cancer in the mammal.

With respect to the above method, in addition to the other methods of the present invention, wherein the method comprises comparing the level of expression of Wip1, a variety of techniques known in the art can be used to compare the level of expression of Wip1. For example, Western blotting can be used to compare the levels of Wip1 protein expressed in two different cell populations. Alternatively, Northern blotting can be used to compare the levels of Wip1 mRNA expressed in two different cell populations. Finally, Southern blotting can be used to compare the number of copies of the Wip1 gene found in two different cell populations. These processes are described in Sambrook et al., Molecular Cloning: A Laboratory Manual, 2^{nd} Ed., Cold Spring Harbor Press, Cold Spring Harbor, N.Y. (1989).

For purposes of the present invention, mammals include, but are not limited to, the order Rodentia, such as mice, and the order Logomorpha, such as rabbits. It is preferred that the mammals are from the order Carnivora, including Felines (cats) and Canines (dogs). It is more preferred that the mammals are from the order Artiodactyla, including Bovines (cows) and Suines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

In a preferred embodiment of the present inventive method of detecting cancer or a predisposition to cancer, Wip1 is in the form of nucleic acid in the test sample and the control sample. The term “nucleic acid” as used herein refers to a chain of nucleotides, as in DNA and RNA. In another preferred embodiment of the present inventive method, Wip1 is in the form of protein in the test sample and the control sample.

The present invention also provides a method of treating cancer in a mammal that expresses the same level or a higher level of Wip1 as compared to a mammal of the same species that does not have cancer. The method comprises administering to the mammal a cancer-treating effective amount of a Wip1 inhibitor. A “cancer-treating effective amount” of a Wip1 inhibitor is an amount sufficient to inhibit the progression of cancer to any degree. It is understood by one of ordinary skill in the art that the inhibition mediated by an inhibitor does not require complete inhibition, as a beneficial or therapeutic effect can be realized with any degree of inhibition. Rather, there are varying degrees of inhibition. In this regard, any suitable inhibitor of Wip1 can be used.

With respect to the present inventive methods, wherein cancer or a predisposition to cancer is either detected or treated, the cancer can be cancer of any tissue from a mammal. For example, the cancer can be leukemia, lymphoma, glioma, breast cancer, bone cancer, pancreatic cancer, small cell lung cancer, lung cancer, brain cancer, skin cancer, melanoma, naso-pharyngeal cancer, stomach cancer, colon cancer, prostate cancer, etc. Preferably, the cancer is cancer of the breast.

In a preferred embodiment of the above inventive method, the Wip1 inhibitor is an oligonucleotide or a morpholino oligomer that inhibits Wip1 by preventing the expression of Wip1 so as to prevent production of either Wip1 mRNA or Wip1 protein, such as by hybridizing to any part of a nucleic acid encoding (i.e., DNA or RNA) Wip1. The oligonucleotide or morpholino oligomer for use in the present inventive method desirably comprises any sequence that is substantially complementary to the sequence of the untranslated region (UTR) of a nucleic acid encoding Wip1 or is substantially complementary to the sequence of a nucleic acid encoding Wip1, such that the oligonucleotide or morpholino oligomer selectively hybridizes to the Wip1 gene or Wip1 mRNA, thereby inhibiting transcription of the gene or translation of the mRNA. It is desirable for the selective hybridization to be highly stringent. In other words, the oligonucleotides or morpholino oligomers specifically hybridize to target sequences of Wip1 mRNA or the Wip1 gene in an amount that is detectably stronger than non-specific hybridization.

Preferably, the oligonucleotide or morpholino oligomer comprises at least 18 genetic nitrogenous bases. More preferably, the oligonucleotide or morpholino oligomer comprises at least 25 genetic nitrogenous bases. Most preferably, the oligonucleotide or morpholino oligomer comprises between about 18 genetic nitrogenous bases and about 25 genetic nitrogenous bases. Preferably, the oligonucleotide or morpholino oligomer hybridizes to exon 1 of a nucleic acid encoding Wip1. In an even more preferred embodiment of the present invention, the oligonucleotide or morpholino oligomer comprises, consists essentially of, or consists of the sequence 5′-CTCCCAGCGAGTACAGCCCCGCCAT-3′ (SEQ ID NO: 1). Alternatively and also preferably, the oligonucleotide or morpholino oligomer hybridizes to any part of an UTR of a nucleic acid encoding Wip1. The oligonucleotide or morpholino oligomer of the present invention can hybridize to any part of the 3′ UTR, which is the region located downstream of the stop codon of the nucleic acid encoding Wip1. Alternatively, the oligonucleotide or morpholino oligomer hybridizes to the 5′ UTR, which is the region located upstream of the initiation codon of the nucleic acid encoding Wip1. In this instance, it is most preferred that the oligonucleotide or morpholino oligomer comprises, consists essentially of, or consists of the sequence 5′-CGGTCCCACGCAGCCCGCCGAATCC-3′ (SEQ ID NO: 2).

The oligonucleotide can comprise at least one modified oligodeoxynucleotide. For example, the oligonucleotide can contain at least one variant linkage that bridges one nucleotide to another, such as a phosphoroamidate linkage or a phosphorothioate linkage instead of the phosphodiester linkages found in unmodified oligonucleotides.

In another preferred embodiment of the present inventive methods, the Wip1 inhibitor is a compound that inhibits the biological function of the Wip1 protein. The compound, for example, blocks Wip1 from binding its substrate, alters the subcellular localization of Wip1, promotes Wip1 degradation, or inhibits Wip1 phosphatase activity. Preferably, the compound is a compound that inhibits Wip1 phosphatase activity. It is to be understood that the inhibition of Wip1 phosphatase activity does not require complete inhibition, as a beneficial or therapeutic effect can be realized with any degree of inhibition. Rather, there are varying degrees of inhibition. Preferably, the compound inhibits at least 10% of Wip1 phosphatase activity. More preferably, the compound inhibits at least 50% of Wip1 phosphatase activity. Most preferably, the compound inhibits 90% or more of Wip1 phosphatase activity. Methods of testing the extent to which a compound inhibits Wip1 phosphatase activity are known in the art and are also described herein (Example 7).

It is furthermore preferred that the compound that inhibits Wip1 phosphatase activity is specific for Wip1, i.e., inhibits the phosphatase activity of Wip1 as opposed to that of another phosphatase. A compound that specifically inhibits Wip1 phosphatase activity can inhibit the phosphatase activity of another phosphatase, but to a significantly lesser extent than the extent to which Wip1 phosphatase activity is inhibited. See Example 7 for methods of testing the specificity of a Wip1 inhibitor compound.

The compound that inhibits Wip1 phosphatase activity preferably is a small molecular weight compound. As used herein, the term “small molecular weight compound” refers to a compound having a molecular weight of less than about 10 kDa as measured by, for example, gel filtration chromatography. One skilled in the art will appreciate that a small molecular weight compound is, generally, a non-peptidic compound that is cell permeable and resistant to degradation. The term “non-peptidic” as used herein refers to not being a protein and not being derived from a protein.

The small molecular weight compound to be used in the present inventive methods can be any small molecular weight compound that inhibits Wip1 phosphatase activity. Suitable small molecular weight compounds include, for example,

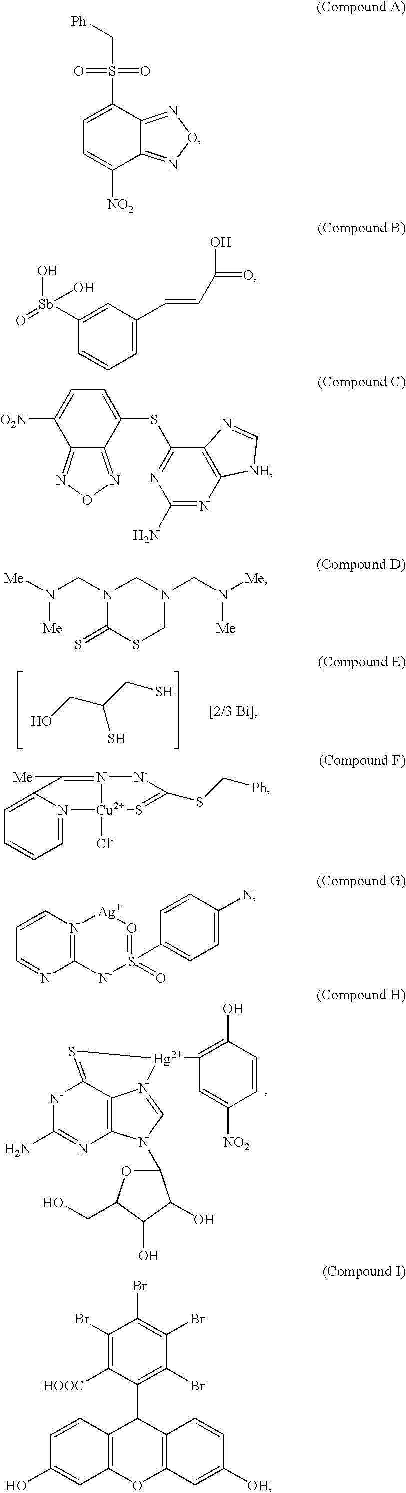

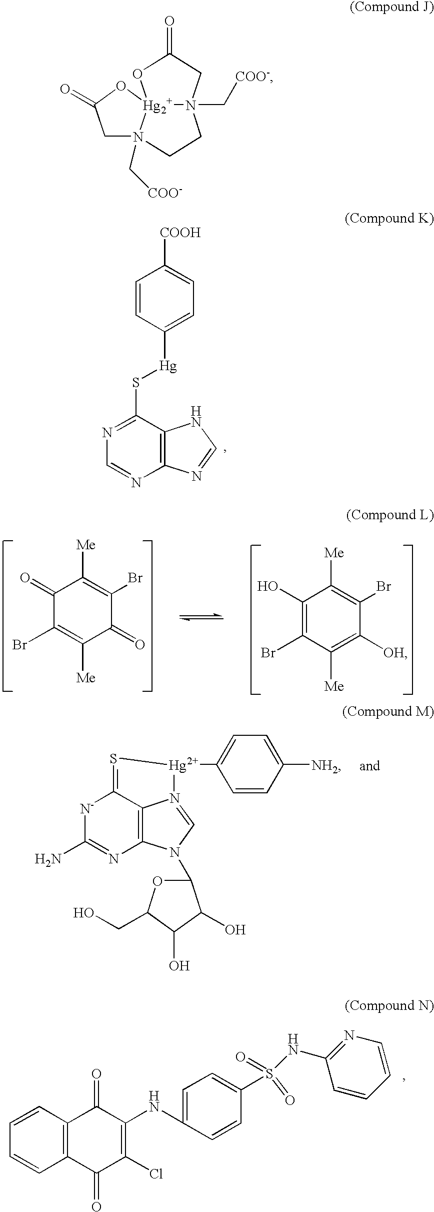

or a pharmaceutically-acceptable salt of any of the foregoing. Preferably, the small molecular weight compound is Compound L or Compound M. These compounds, as well as salts thereof, are also provided by the present invention.

The compounds discussed herein can be in the form of a salt, which is preferably a pharmaceutically-acceptable salt. Suitable pharmaceutically-acceptable acid addition salts include non-toxic inorganic or organic acid addition salts of the base compounds. Illustrative inorganic acids which form suitable salts include hydrochloric, hydrobromic, sulfuric, phosphoric acids, metaphosphoric, and nitric acids. The salts can be in the form of acid metal salts, such as sodium monohydrogen orthophosphate and potassium hydrogen sulfate. Illustrative organic acids which form suitable salts include the mono, di, and tricarboxylic acids. Illustrative of such acids are, for instance, acetic, glycolic, lactic, pyruvic, malonic, succinic, glutaric, flimaric, malic, tartaric, citric, ascorbic, maleic, hydroxymaleic, benzoic, hydroxybenzoic, pheylacetic, cinnamic, salicylic, and 2-phenoxybenzoic acids. Other organic acids which form suitable salts are the sulfonic acids (e.g., methane sulfonic acid), gluconic, arylsulphonic acids (e.g., p-toluenesulphonic acid), and 2-hydroxyethane sulfonic acid. These salts and base compounds can exist in either a hydrated or a substantially anhydrous form. The acid salts are prepared by standard techniques, such as by dissolving the free base in aqueous or aqueous-alcohol solution or other suitable solvent containing the appropriate acid and isolating the salt by evaporating the solution, or by reacting the free base in an organic solvent in which case the salt separates directly or can be obtained by concentration of the solution. In general, the acid addition salts of the compounds of this invention are crystalline materials, which are soluble in water and various hydrophilic organic solvents and which, in comparison to their free base forms, demonstrate higher melting points and an increased stability.

Methods of making such small molecular weight compounds are known in the art. Alternatively, the compounds can be obtained by purchasing from companies or institutions, such as from the Diversity Set Library maintained by the National Cancer Institute (http://dtp.nci.nih.gov/branches/dscb/diversity_explanation.html).

With respect to the present inventive method of treating cancer, the mammal can be any mammal as discussed herein. Preferably the mammal is a human. However, the mammal should also be one that expresses the same level or a higher lever of Wip1 as compared to a mammal of the same species that does not have cancer. Methods of assaying the expression level of Wip1 are known in the art and include, for instance, Western blotting, Southern blotting, Northern blotting, quantitative PCR, and the like (see, for instance, Sambrook et al., Molecular Cloning: A Laboratory Manual, 2^{nd} Ed., Cold Spring Harbor Press, Cold Spring Harbor, N.Y. (1989), and Examples 1-3 set forth below).

A method of inhibiting Wip1 in a cell is further provided by the present invention. The method comprises administering to the cell a compound selected from the group consisting of Compound A, Compound B, Compound C, Compound D, Compound E, Compound F, Compound G, Compound H, Compound I, Compound J, Compound K, Compound L, Compound M, and Compound N, or a pharmaceutically-acceptable salt of any of the foregoing, in an amount effective for inhibiting Wip1 in a cell. Preferably, Compound L or Compound M is the compound.

With respect to the present inventive methods, the cell can be a cell from any tissue of any living system. The cell can be a cell of a cultured cell line or a cell obtained from a host, e.g., a mammal. Preferably, the cell is located within the host, and the corn pound is administered to the host.

For purposes herein, the host can be any host, including all prokaryotic species and eukaryotic species. If the host is prokaryotic, then the cell is the prokaryotic cell and not in the prokaryote. Prokaryotic cells include any cell that lacks a membrane-bound nucleus, such as bacterial cells. Eukaryotic cells include cells of yeast, fingi, plants, algae, birds, reptiles, and mammals. Preferably, the host is a mammal. For purposes of the present invention, mammals include, but are not limited to, those that are discussed herein. An especially preferred mammal is the human.

In a preferred embodiment of the present inventive method of inhibiting Wip1 in a cell, a host of which the cell is comprised is afflicted with a disease or a condition, either of which is associated with Wip1 overexpression, and the method effectively treats the disease or condition. The phrase “associated with Wip1 overexpression” as used herein refers to a disease or condition in which higher levels of Wip1 protein or nucleic acid, e.g. MRNA or DNA, correlate with the disease or condition. The phrase “higher levels” as used herein refers to an amplification of Wip1 nucleic acid or an overexpression of Wip1 protein in comparison to a matched control. The disease or condition can be, for example, a benign tumor, e.g., a cyst, fibroid, a polyp, and the like. The disease, which afflicts the host, can be a cancer, such as leukemia, lymphoma, glioma, breast cancer, bone cancer, pancreatic cancer, small cell lung cancer, lung cancer, brain cancer, skin cancer, melanoma, naso-pharyngeal cancer, stomach cancer, colon cancer, prostate cancer, etc. Preferably, the cancer is breast cancer.

As used herein, the terms “treat” and “inhibit” and words stemming therefrom, do not necessarily imply a complete treatment or inhibition. Rather, there are varying degrees of treatment or inhibition of which one of ordinary skill in the art recognizes as having a potential benefit or therapeutic effect. In this respect, the cancer can be treated to any extent through the present inventive methods. For example, at least 10% of the growth of a tumor or the metastasis of the cancer desirably is inhibited upon administration of a compound selected from Compounds A-N. Preferably, at least 50% of the growth of a tumor or the metastasis of the cancer is inhibited upon administration of a compound selected from Compounds A-N. More preferably, at least 90% of the growth of a tumor or the metastasis of the cancer is inhibited upon administration of a compound selected from Compounds A-N. Furthermore, in this regard, the Wip1 phosphatase activity in a cell can be inhibited to any level through the present inventive methods. Preferably, at least 10% of the Wip1 phosphatase activity in a cell is inhibited upon administration of a compound selected from Compounds A-N. It is more preferred that at least 50% of the Wip1 phosphatase activity in a cell is inhibited upon administration of a compound selected from Compounds A-N. Most preferably, at least 90% or more Wip1 phosphatase activity in a cell is inhibited upon administration of the compound.

For purposes of the present inventive methods, the Wip1 inhibitor can be administered in a variety of forms. For example, when the Wip1 inhibitor is an oligonucleotide or morpholino oligomer, the oligonucleotide or morpholino oligomer can be administered in the form of a liposome. Alternatively, the oligonucleotide or morpholino oligomer can be administered in the form of a vector. One of ordinary skill in the art will appreciate that any of a number of vectors known in the art are suitable for use in the invention. Examples of suitable vectors include, for instance, plasmids, plasmid-liposome complexes, and viral vectors, e.g., parvoviral-based vectors (i.e., adeno-associated virus (AAV)-based vectors), retroviral vectors, herpes simplex virus (HSV)-based vectors, and adenovirus-based vectors. Any of these expression constructs can be prepared using standard recombinant DNA techniques described in, e.g., Sambrook et al. (1989), supra and Ausubel et al., Current Protocols in Molecular Biology, Greene Publishing Associates and John Wiley & Sons, New York, N.Y. (1994).

Alternatively, the Wip1 inhibitor can be a part of a composition, such as a pharmaceutical composition. In this regard, the present invention provides a pharmaceutical composition comprising a compound selected from the group consisting of Compound A, Compound B, Compound C, Compound D, Compound E, Compound F, Compound G, Compound H, Compound I, Compound J, Compound K, Compound L, Compound M, and Compound N, or a pharmaceutically-acceptable salt of any of the foregoing, and a pharmaceutically-acceptable carrier. The pharmaceutical composition can comprise more than one active ingredient, such as more than the Wip1 inhibitor, e.g., a morpholino oligomer of the present invention and Compound L, or can comprise a Wip1 inhibitor in combination with another pharmaceutically active agent or drug, i.e., one that is not a Wip1 inhibitor.

The composition can comprise a carrier, which can be any suitable carrier. Preferably, the carrier is a pharmaceutically-acceptable carrier. With respect to pharmaceutical compositions, the carrier can be any of those conventionally used and is limited only by chemico-physical considerations, such as solubility and lack of reactivity with the active compound(s), and by the route of administration. It will be appreciated by one of skill in the art that, in addition to the following described pharmaceutical composition, the inhibitors of the present inventive methods can be formulated as inclusion complexes, such as cyclodextrin inclusion complexes, or liposomes.

The pharmaceutically-acceptable carriers described herein, for example, vehicles, adjuvants, excipients, and diluents, are well-known to those skilled in the art and are readily available to the public. Typically, the pharmaceutical composition comprising the carrier and the oligonucleotide or morpholino oligomer can comprise a physiological saline solution; dextrose or other saccharide solution; or ethylene, propylene, polyethylene, or other glycol. It is preferred that the pharmaceutically-acceptable carrier is one which is chemically inert to the active compound(s) and one which has no detrimental side effects or toxicity under the conditions of use.

The choice of carrier will be determined in part by the particular Wip1 inhibitor, as well as by the particular method used to administer the inhibitor. Preferably, the oligonucleotides or morpholino oligomers are administered with the carrier ethoxylated polyethylenimine (EPEI), which is a weakly basic delivery reagent that electrostatically binds to the anionic charges of oligonucleotides and morpholino/oligonucleotide duplexes. See Morcos, Genesis 30: 94-102 (2001), for more description on this method. Accordingly, there are a variety of suitable formulations of the pharmaceutical composition. The following formulations for topical, oral, aerosol, parenteral, subcutaneous, intravenous, intramuscular, interperitoneal, rectal, and vaginal administration are exemplary and are in no way limiting. One skilled in the art will appreciate that these administration routes are known. Although more than one route can be used to administer a particular inhibitor, a particular route can provide a more immediate and more effective response than another route. If, for example, the cancer is in the form of a tumor, preferably the Wip1 inhibitor is administered peritumorally or intratumorally.

Injectable formulations are among those formulations that are preferred in accordance with the present invention. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)).

Topical formulations are well-known to those of skill in the art. Such formulations are particularly suitable in the context of the present invention for application to the skin.

Formulations suitable for oral administration can consist of (a) liquid solutions, such as an effective amount of the inhibitor dissolved in diluents, such as water, saline, or dextrose solutions; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically-acceptable surfactant. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

The Wip1 inhibitors, alone, in combination with another Wip1 inhibitor, or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer. Such spray formulations also may be used to spray mucosa.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The Wip1 inhibitors can be administered in a physiologically-acceptable pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically-acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts. Suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-b-aminopropionates, and 2-alkyl-imidazoline quaternary ammonium salts, and (e) mixtures thereof.

The parenteral formulations will typically contain from about 0.5% to about 25% by weight of the active ingredient in solution. The parenteral formulations can contain preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Additionally, the present inventive Wip1 inhibitors, or compositions comprising a Wip1 inhibitor, can be made into suppositories by mixing with a variety of bases, such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

One of ordinary skill in the art will readily appreciate that the Wip1 inhibitors can be modified in any number of ways, such that the therapeutic efficacy of the inhibitor is increased through the modification. For instance, the compound can be conjugated either directly or indirectly through a linker to a targeting moiety. The practice of conjugating inhibitors to targeting moieties is known in the art. See, for instance, Wadwa et al., J. Drug Targeting 3: 111 (1995), and U.S. Pat. No. 5,087,616. The term “targeting moiety” as used herein refers to any molecule or agent that specifically recognizes and binds to a cell-surface receptor, such that the targeting moiety directs the delivery of the inhibitor to a population of cells on which surface the receptor is expressed. Targeting moieties include, but are not limited to, antibodies, or fragments thereof, peptides, hormones, growth factors, cytokines, and any other naturally- or non-naturally-existing ligands, which bind to cell surface receptors. The term “linker” as used herein, refers to any agent or molecule that bridges the inhibitor to the targeting moiety. One of ordinary skill in the art recognizes that sites on the compound, which are not necessary for the function of the compound, are ideal sites for attaching a linker and/or a targeting moiety, provided that the linker and/or targeting moiety, once attached to the inhibitor, do(es) not interfere with the function of the inhibitor, i.e., the ability to inhibit Wip1 in a cell, or treat cancer in a mammal.

Alternatively, the Wip1 inhibitors can be modified into a depot form, such that the manner in which the inhibitor is released into the body to which it is administered is controlled with respect to time and location within the body (see, for example, U.S. Pat. No. 4,450,150). Depot forms of inhibitors can be, for example, an implantable composition comprising the inhibitor and a porous material, such as a polymer, wherein the inhibitor is encapsulated by or diff-used throughout the porous material. The depot is then implanted into the desired location within the body, and the inhibitor is released from the implant at a predetermined rate by diffusion through the porous material.

The Wip1 inhibitors can be administered to the cell in vitro. As used herein, the term “in vitro” means that the cell to which the inhibitor is being administered is not in a living organism. The Wip1 inhibitors alternatively can be administered to the cell in vivo. As used herein, the term “in vivo” means that the cell is a part of a living organism or is the living organism. The Wip1 inhibitors can be administered to a host, e.g., a mammal, ex vivo, wherein the inhibitor is administered to cells in vitro and the cells are subsequently administered to the host.

For purposes of all of the present inventive methods, the amount or dose of the Wip1 inhibitor administered should be sufficient to effect the desired, e.g., a therapeutic, response over a reasonable time frame. Particularly, the dose of the Wip1 inhibitor should be sufficient to inhibit Wip1 phosphatase activity in a cell or to treat cancer in a host within about 1-2 hours, if not 3-4 hours, from the time of administration. The dose will be determined by the efficacy of the particular Wip1 inhibitor and the condition of the animal (e.g., human), as well as the body weight of the animal (e.g., human) to be treated. Many assays for determining an administered dose are known in the art. For example, an assay, which comprises comparing the extent to which the phosphatase activity of a Wip1 protein is inhibited in a cell upon administration of a given dose of a Wip1 inhibitor to a mammal among a set of mammals that are each given a different dose of the inhibitor, could be used to determine a starting dose to be administered to a mammal. The extent to which the phosphatase activity of the Wip1 protein is inhibited upon administration of a certain dose can be assayed as described herein in Example 7.

The size of the dose also will be determined by the existence, nature, and extent of any adverse side effects that might accompany the administration of a particular Wip1 inhibitor. Ultimately, the attending physician will decide the dosage of the Wip1 inhibitor with which to treat each individual patient, taking into consideration a variety of factors, such as age, body weight, general health, diet, sex, inhibitor to be administered, route of administration, and the severity of the condition being treated.

The Wip1 inhibitor, such as the oligonucleotides, morpholino oligomers, or compounds A-N of the present invention, can be administered alone or in combination with other suitable components. Such components include other active agents, such as anti-cancer agents and agents that help the Wip1 inhibitor inhibit expression of Wip1 protein or Wip1 mRNA or inhibit Wip1 phosphatase activity more effectively.

A method of screening an oligonucleotide or morpholino oligomer for the ability to inhibit the expression of Wip1 is further provided by the present invention. The method comprises comparing the level of expression of Wip1 in a test sample obtained from Wip1-expressing cells that have been contacted with the oligonucleotide or morpholino oligomer to the level of expression of Wip1 in a control sample obtained from Wip1-expressing cells that have not been contacted with the oligonucleotide or morpholino oligomer. In this method, a lower level of expression of Wip1 in the test sample as compared to the control sample is indicative of the ability of the oligonucleotide or morpholino oligomer to inhibit the expression of Wip1.

Further provided by the present invention is a method of determining the efficacy with which a test oligonucleotide or morpholino oligomer inhibits Wip1 expression. The method comprises comparing the level of expression of Wip1 in a test sample obtained from Wip1-expressing cells that have been contacted with the test oligonucleotide or morpholino oligomer to the level of expression of Wip1 in a control sample obtained from Wip1-expressing cells that have been contacted with an oligonucleotide or morpholino oligomer that is known to inhibit the expression of Wip1. A lower level of expression of Wip1 in the test sample as compared to the control sample is indicative of the test oligonucleotide or morpholino oligomer having a greater efficacy for inhibiting the expression of Wip1 than the known oligonucleotide or morpholino oligomer, whereas a higher level of expression of Wip1 in the test sample as compared to the control sample is indicative of the test oligonucleotide or morpholino oligomer having a lower efficacy for inhibiting the expression of Wip1 than the known oligonucleotide or morpholino oligomer.

Wip1 can be in the form of nucleic acid in the test sample and the control sample. Alternatively, Wip1 can be in the form of protein in the test sample and the control sample.

With respect to the method of screening an oligonucleotide or morpholino oligomer and the method of determining the efficacy with which a test oligonucleotide or morpholino oligomer inhibits Wip1 expression, a variety of techniques known in the art can be used to compare the level of expression of Wip1. For example, Western blotting can be used to compare the levels of Wip1 protein expressed in two different cell populations. Alternatively, Northern blotting can be used to compare the levels of Wip1 mRNA expressed in two different cell populations. Finally, Southern blotting can be used to compare the number of copies of the Wip1 gene found in two different cell populations. These processes are described in Sambrook et al. (1989), supra.

The present invention also provides a method of screening a compound for Wip1-inhibiting activity. The method comprises comparing the level of Wip1 phosphatase activity in a test sample that has been contacted with the compound to the level of Wip1 phosphatase activity in a control sample that has not been contacted with the compound. A lower level of Wip1 phosphatase activity in the test sample as compared to the control sample is indicative of the ability of the compound to inhibit Wip1. Example 7, set forth below, describes a particular embodiment of the inventive method.

A method of determining the efficacy with which a test compound inhibits Wip1 is also provided by the present invention. The method comprises comparing the level of Wip1 phosphatase activity in a test sample that has been contacted with the test compound to the level of Wip1 phosphatase activity in a control sample that has been contacted with a compound that is known to inhibit Wip1. In this method, a lower level of Wip1 phosphatase activity in the test sample as compared to the control sample is indicative of the test compound having a greater efficacy for inhibiting Wip1 than the known compound, whereas a higher level of Wip1 phosphatase activity in the test sample as compared to the control sample is indicative of the test compound having a lower efficacy for inhibiting Wip1 than the known compound.

A variety of techniques known in the art can be used to compare levels of phosphatase activity. An example of a Wip1 phosphatase activity assay can be found in Fiscella et al. (1997), supra, and Example 7 set forth below.

In the present inventive methods of screening a compound for Wip1-inhibiting activity and of determining the efficacy with which a test compound inhibits Wip1, the test sample and/or the control sample can be obtained from Wip1-expressing cells. The cells can endogenously express Wip1 or the cells can exogenously express Wip1. Cells that exogenously express Wip1 can be obtained through methods known in the art and are also described herein in Example 4 and Example 7. Cells that endogenously express Wip1 include those that are described in Example 1.

### EXAMPLES

These examples further illustrate the invention but, of course, should not be construed as in any way limiting its scope.

For convenience, the following abbreviations are used herein: Wip1, wild-type p53-induced phosphatase; UV, ultra-violet; PPMD1, protein phosphatase, magnesium-dependent 1; FISH, fluorescent in situ hybridization; mRNA, messenger RNA; UTR, untranslated region; HSV, herpes simplex virus; AA V, adeno-associated virus; EPEI, ethoxylated polyethylenimine; GAPDH, glyceraldehyde 3-phosphate dehydrogenase; GADD34, growth arrest and DNA damage-inducible 34; and PCR, polymerase chain reaction; cDNA, complementary DNA; BAC, bacteria artificial chromosome; dUTP, deoxyuridine triphosphate; MEF, mouse embryo fibroblasts.

The following references, to the extent that they provide exemplary procedural or other details supplementary to those set forth herein, are specifically incorporated herein by reference:

Birren et al., Genome Analysis: A Laboratory Manual Series, Volume 1, Analyzing DNA, Cold Spring Harbor Laboratory Press, Cold Spring Harbor, N.Y. (1997),

Birren et al., Genome Analysis: A Laboratory Manual Series, Volume 2, Detecting Genes, Cold Spring Harbor Laboratory Press, Cold Spring Harbor, N.Y. (1998),

Birren et al., Genome Analysis: A Laboratory Manual Series, Volume 3, Cloning Systems, Cold Spring Harbor Laboratory Press, Cold Spring Harbor, N.Y. (1999),

Birren et al., Genome Analysis: A Laboratory Manual Series, Volume 4, Mapping Genomes, Cold Spring Harbor Laboratory Press, Cold Spring Harbor, N.Y. (1999),

Harlow et al., Antibodies: A Laboratory Manual, Cold Spring Harbor Laboratory Press, Cold Spring Harbor, N.Y. (1988),

Harlow et al., UsingAntibodies: A Laboratory Manual, Cold Spring Harbor Laboratory Press, Cold Spring Harbor, N.Y. (1999),

Hoffman, Cancer and the Search for Selective Biochemical Inhibitors, CRC Press (1999),

Pratt, The Anticancer Drugs, 2nd edition, Oxford University Press, NY (1994),

QIAexpress Detection and Assay Handbook, 2nd edition, QIAGEN Inc., 28159 Avenue Stanford, Valencia, Calif. 91355 (April 1999), and Sambrook et al. (1989), supra.

### Example 1

This example demonstrates the expression levels of Wip1 mRNA in human tumor cell lines and tumor samples.

Total RNA was extracted from 67 human tumor cell lines of which 58 are listed in O'Connor et al., Cancer Research 57: 4285-4300 (1997) and 11 are IMR-90, NDA-N, Sum 52, NCI-ADR, BT-474, MCF7, MCI-H%22, ACHN, CAKI-1, MOLT4, and OVCAR4, in addition to 11 tumor samples (National Cancer Institute, National Institutes of Health), using the RNeasy kit (Qiagen, Inc., Valencia, Calif.). Wip1 mRNA levels in primary breast tumors were determined after reverse transcription coupled to the Real-Time PCR procedure using an ABI PRISM® 7700 Sequence Detection System (Applied Biosystems, Foster City, Calif.) and the SYBR Green PCR Master Mix (Applied Biosystems, Foster City, Calif.). Expression levels were determined relative to the expression of GAPDH and GADD34. The following primer pairs were used: WIP1: 5′-tgcccgggagcacttgt-3′ (SEQ ID NO: 3)/ 5′-ggcagcgcaaaccttagc-3′ (SEQ ID NO: 4) and 5′-gacccgaaggatgactttgtc-3′ (SEQ ID NO: 5)/ 5′-gcttctgagggtcaagagtg-3′ (SEQ ID NO: 6); GADD34, 5′-cctctacttctgccttgtctcca-3′ (SEQ ID NO: 7)/ 5′-ccgtggcttgattctcttcct-3′ (SEQ ID NO: 8); and GAPDH, 5-gaaggtgaaggtcggagtc-3′ (SEQ ID NO: 9)/ 5′-gaagatggtgatgggatttc-3′ (SEQ ID NO: 10). mRNA levels in the panel of human tumor cell lines were analyzed using a dot-blotting procedure described in Koch-Paiz et al., Biotechniques 29: 706-714 (2000). polyU served as a control for relative mRNA content.

In the human tumor cell lines tested, Wip1 mRNA levels were 4.7 to 9.4 times higher in the four breast tumor cell lines MDA-MB361, BT474, MCF-7 and KPL-1 as compared to IMR-90 cells. This analysis demonstrated that Wip1 mRNA was overexpressed in tumor samples.

### Example 2

This example demonstrates that WIP1 is amplified in some breast cancer cell lines.

Genomic DNA from IMR-90, NDA-N, Sum52, NCI-ADR, BT-474, MCF7, MCI-H%22, ACHN, CAKI-1, MOLT4, and OVCAR4 cell lines were digested with PvuII enzyme (New England Biolabs, Beverly, Mass.) The digested DNA was Southern blotted according to Sambrook et al. (1989), supra, using probes comprising the full-length cDNA sequence of Wip1, which has the GenBank accession number, U78305. From this analysis, it was shown that MCF7 and BT-474 breast cancer cell lines, and not the other cell lines tested, overexpressed WIP1. This analysis demonstrated that Wip1 mRNA was overexpressed in breast cancer.

### Example 3

This example demonstrates the amplification of WIP1 in primary tumors.

The BLASTN program was used to localize the WIP1 gene to three overlapping bacteria artificial chromosome (BAC)clones (RP11-15E18, RP11-634F5, and RP11-1081E4) in the draft human genome sequence that map to 17q23. BAC clone RP11-634F5, representing WIP1, labeled with SpectrumOrange-deoxyuridine triphosphate (dUTP) (Applied Biosystems, Foster City, Calif.) and centromere specific, SpectrumGreen-dUTP-labeled chromosome 17 probe (Applied Biosystems, Foster City, Calif.) were hybridized to a tissue microarray containing primary breast tumors (Kononen et al., Nature Medicine 4: 844-847 (1998)). Nuclei were stained with 4′, 6-diamidino-2-phenylindole (Sigma-Aldrich, St. Louis, Mo.). Tumor samples with at least a 3-fold increase in the number of WIP1 signals, as compared with chromosome 17 centromere signals, were considered to be amplified. Accordingly, 37 of the 326 (11.3%) tumors tested had WIP1 region amplified. From this example, it is evident that the WIP1 is amplified in some primary tumors.

### Example 4

This example demonstrates the effect of WIP1 overexpression on p38-mediated phosphorylation of p53.

IMR-90 cells were infected with plasmids encoding Wip1, which were made by cloning the Wip1 cDNA into the PINCO vector (Grignani, et al., Cancer Research 58: 14-19 (1998)) using BamHI/NotI sites, and with H-RasV12, which was obtained from S. Lowe (Cold Spring Harbor Laboratory, Cold Spring Harbor, N.Y.). p53 was immunoprecipitated from 1 mg of the total protein extract from these cells, and phosphorylation on Ser33 or Ser46 of p53 was assessed by Western blotting with antibodies specific for phosphorylated p53 as described in Sakaguchi et al., Genes Development 12: 2831-2841 (1998). In cells that overexpressed Wip1, p53 had little, if any, phosphorylation on Ser33 or Ser46 as compared to cells infected with H-RasV12 alone. From this example, it is evident that the overexpression of WIP1 results in less phosphorylation of p53 on sites phosphorylated by p38 kinase.

### Example 5

This example demonstrates that WIP1 phosphatase complements different oncogene proteins for growth in soft agar.

Retroviruses containing WIP1 and different oncogenes, including H-RasV12, MYC or NEU1, were co-infected into wild-type mouse embryo fibroblasts (MEFs) and were analyzed for anchorage-independent growth and the ability to form foci in soft agar. The IRES c-myc (human) retroviral vector, pBabeMNIRESgfpmyc (Oster et al., Molecular and Cellular Biology 20: 6768-6778 (2000)), was kindly provided by L. Z. Penn (Toronto University, Toronto, Ontario); pBabe-c-neu (Bargmann et al., Cell 45: 649-657 (1986)) (rat) was obtained from P. Sicinski (Dana-Farber Cancer Institute, Boston, Mass.). Suppression of colony formation was determined 2-3 weeks after infection of MEFs in 100 mm dishes with the designated retroviruses. Colonies were selected with 500 μg/ml G418 (Sigma-Aldrich, St. Louis, Mo.), and fixed and stained with 0.1% crystal violet (Sigma-Aldrich, St. Louis, Mo.). For soft agar cloning, 20,000 puromycin-selected, infected cells were seeded in 0.5% agar into each well of 6-well plates. Samples were analyzed in triplicate. The results of this assay are shown below in Table 1.

TABLE 1

| MEF type | Retrovirus | Growth in soft agar |
|---|---|---|
| Wild-type | Puro | − |
| | H-rasV12 | − |
| | neu | − |
| | myc | − |
| | Wip1 | − |
| | H-ras V12 + Wip1 | + |
| | neu + Wip1 | + |
| | myc + Wip1 | + |
| | ras + myc | ++ |
| | ras + neu | + |
| p53^{-/-} | Puro | − |
| | H-ras V12 | ++ |
| | Wip1 | − |
| | H-ras V12 + Wip1 | ++ |
| | Neu | +++ |
| | myc | +++ |

Score:
+ = 10-30 colonies;
++ = 30-100 colonies;
+++ = more than 100 colonies per well of a 6-well plate.

In both assays, WIP1 complemented H-RasV12 for transformation of wild-type MEFs. From this analysis, it is evident that WIP1 is a proto-oncogene.

### Example 6

This example demonstrates a method of delivering morpholino oligomers to cells in culture.

Morpholino oligomers comprising the sequence of SEQ ID NO: 1 or SEQ ID NO: 2 were synthesized by and purchased from Gene Tools (Philomath, Oreg.). Sterile water (600 microliters (μl)) was added to one vial containing 300 nM of Special Delivery morpholino/DNA (Gene Tools, Philomath, Oreg.) to make a 0.5 millimolar (mM) stock solution. Sterile water (200 μl) was added to 100 nM Special Delivery Standard Control morpholino oligomers (Gene Tools, Philomath, Oreg.) to make a 0.5 stock solution. In a 15 mililiter (ml) centrifuge tube, sterile water (188.8 μl) was mixed with 5.6 μl of the 0.5 mM Special Delivery morpholino oligomer/DNA stock solution and 5.6 μl of 200 μM EPEI Special Delivery solution (Gene Tools, Philomath, Oreg.) and vortexed immediately upon mixing. The mixture was then incubated at room temperature for exactly 20 minutes. Serum-free medium (1.8 ml) was added to the mixture and vortexed immediately upon addition. This mixture (500 μl) was then added to media-free cells and incubated in an incubator for 3 hours. Afterwards, the mixture was removed from the cells via centrifugation and fresh media containing serum was added to the cells. The cells were incubated for at least 16 hours before testing for delivery of the morpholino oligomers. This example demonstrates a method of delivering morpholino oligomers to cultured cells.

### Example 7

This example demonstrates a method of screening for Wip1 inhibitors and the identification of effective Wip1 inhibitors.

The expression plasmid pET14bWip1 was constructed by common subcloning techniques and propagated in the DH5cc (Invitrogen, Carlsbad, Calif.) strain of E. coli. In brief, the polymerase chain reaction was used to amplify the segment of the human Wip1 gene, encoding the full-size Wip1 enzyme (Bulavin et al., Nature Genetics 31: 210-215 (2002)). The 5′ end amplification primer contained the Nde1 restriction site followed by an initiating methionine codon for residue Met. The 3′ end primer contained a stop codon immediately adjacent to the natural Wip1 last codon, followed by BamHI restriction site. The polymerase chain reaction product was digested with Ndel-BamHI and cloned into the plasmid pET14b (Novagen, San Diego, Calif.). The resulting plasmids, called pET14bWip1, carried the coding sequences for a full-size Wip1 enzyme with His-Tag on the NH₂ terminus. The cloned fragment was sequenced to confirm proper construction of the initiation signal and to ensure that no other mutations were introduced. Protein was overexpressed in E. coli BL21(DE3) pLysS cells (Novagen) and purified according to His-Bind Kits (Novagen) protocol.

Phosphatase activity of Wip1 was monitored with IQ™ Phosphatase Assay Kit (Pierce, Rockford, Ill.). IQ™ Technology is a homogeneous assay platform based on fluorescence intensity quenching. The assay incorporates an iron-containing compound that has the capacity to bind to phosphoryl groups present on fluorescent dye-labeled phosphorylated peptides. At the end of phosphatase reaction, the labeled peptide population contains a mixture of phosphorylated and nonphosphorylated peptide species. The iron-containing compound binds specifically to the phosphorylated peptide present and its fluorescence is quenched. The observed relative fluorescence units increase in proportion to the extent of dephosphorylation of the phosphopeptide. The standard phosphatase assay was performed by incubating 0.2 μg recombinant Wip1 in the buffer, containing 20 mM MOPS, pH 7.0, 20 mM MgCl₂, and 30 μM of phosphoserine peptide substrate (LRRApSLG) at 30° C. for 1 hour. The reaction was terminated by addition of 60 μl IQ™ Working Solution. Fluorescent intensity was measured on the Fluorometer at 560/590 ex/em. Compounds (2 ml) from a chemical library of potential Wip1 inhibitors dissolved in 50% DMSO were added to fmal concentration of 10, 1, 0.5, or 0.2 μM. Control reactions contained only 6% DMSO.

The diversity set was derived from the almost 140,000 compounds available on plates. Only compounds for which at least one gram of material was available were considered. This was done to allow a large number of copies to be made. The 71,756 compounds meeting this criterion were then reduced to the final set using the program Chem-X (Oxford Molecular Group). Chem-X uses defined centers (hydrogen bond acceptor, hydrogen bond donor, positive charge, aromatic, hydrophobic, acid, base) and defined distance intervals to create a particular finite set of pharmacophores. Three-point pharmacophores were used with the default settings, resulting in almost 1,000,000 possible pharmacophores. The Chem-X diverse subset generating function reads through a set of structures and for every structure, determines the acceptable conformations of that structure. For each acceptable conformation, it determines all the pharmacophores for that conformation. The pharmacophores for the current structure are compared to the set of all pharmacophores found in structures already accepted into the diverse subset. If the current structure has more than a preset number of new pharmacophores, it is added to the diverse subset. The requirements were set as 5 new pharmacophores and, additionally, 5 or fewer rotatable bonds. Because the selection. procedure is order dependent, the order in which the structures were considered was randomized. This procedure resulted in the selection of 1990 compounds.

Wip1 activity was measured in the presence of 1990 chemical compounds, 10 μM final concentration each. Among them, 14 compounds were found that completely inhibited the activity of enzyme. The effectiveness of these compounds was monitored at the concentration of1 μM, 0.5 μM, and 0.25 μM (Table 2).

TABLE 2

Wip1 Activity
(light units @ 590 nm)

| Compound | 10 μm | 1 μm | 0.5 μm | 0.25 μm |
|---|---|---|---|---|
| DMSO (control) | 1400 | 725 | 740 | 740 |
| A | 0 | 250 | 725 | 745 |
| B | 0 | 575 | 723 | 740 |
| C | 0 | 610 | 750 | 745 |
| D | 0 | 595 | 740 | 750 |
| E | 0 | 605 | 740 | 745 |
| F | 0 | 100 | 695 | 740 |
| G | 0 | 440 | 741 | 740 |
| H | 0 | 225 | 690 | 675 |
| I | 0 | 250 | 710 | 625 |
| J | 0 | 260 | 750 | 630 |
| K | 0 | 300 | 680 | 630 |
| L | 0 | 60 | 220 | 375 |
| M | 0 | 145 | 400 | 525 |
| N | 0 | 645 | 710 | 550 |

As is evident from the data of Table 2, at the concentration of 0.25 μM, the most effective inhibitors were Compounds L and M. Specificity was estimated by comparing the activities of Wip1 and phosphatase PP2A in the presence of Compound L at 1 μM final concentration. The activity of Wip1 was nearly not detectable, while the activity of PP2A decreased only by 5% (Table 3).

TABLE 3

Phosphatase Activity
(light units @ 590 nm)

| Phosphatase | DMSO (control) | Compound L |
|---|---|---|
| Wip1 | 500 | 5 |
| PP2A | 760 | 690 |

This example demonstrated that Compounds L and M were the most effective at inhibiting Wip1.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of treating cancer in a mammal that expresses the same level or a higher level of Wip1 as compared to a mammal of the same species that does not have cancer, which method comprises administering to the mammal a cancer-treating effective amount of a Wip1 inhibitor, whereupon the mammal is treated for cancer.

2. The method of claim 1, wherein the inhibitor is a compound that inhibits Wip1 phosphatase activity.

3. The method of claim 2, wherein the inhibitor is a compound having a molecular weight of about 10 kDa or less.

4. The method of claim 3, wherein the compound is selected from the group consisting of 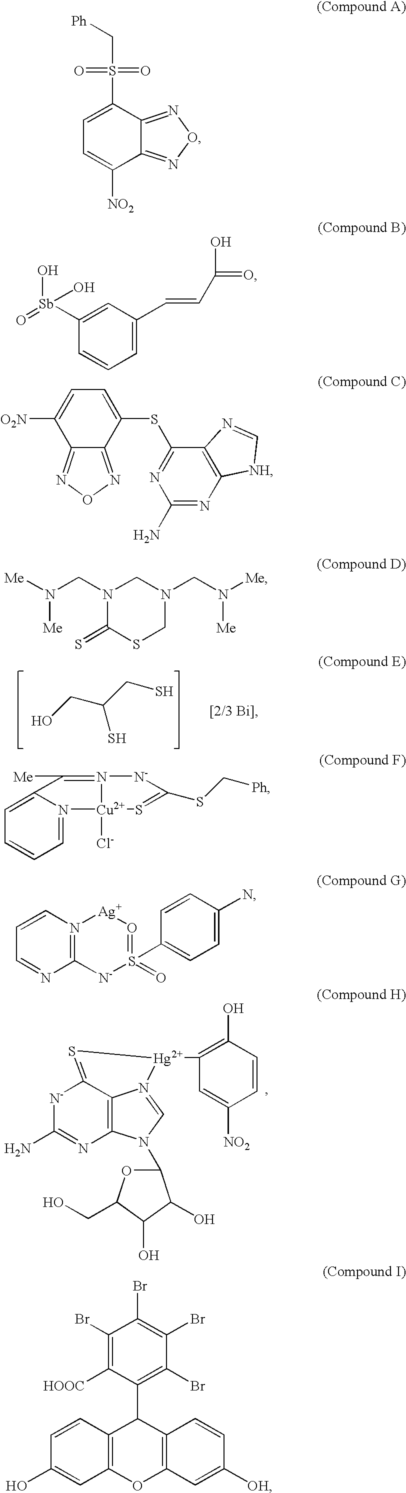 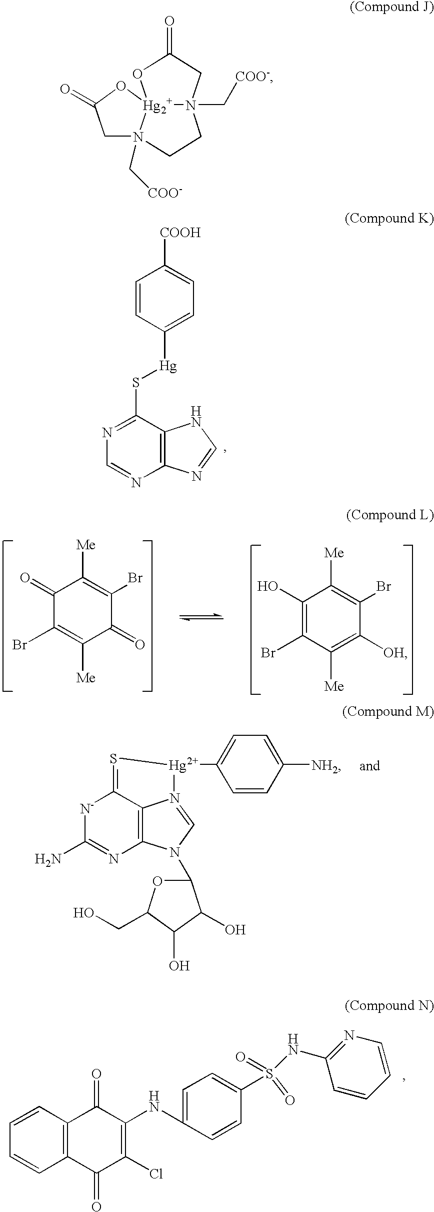
or a pharmaceutically-acceptable salt of any of the foregoing.

5. The method of claim 4, wherein the compound is Compound L.

6. The method of claim 4, wherein the compound is Compound M.

7. The method of claim 1, wherein the cancer is breast cancer.

8. A method of inhibiting Wip1 phosphatase activity in a cell, which method comprises administering to the cell a compound selected from the group consisting of Compound A, Compound B, Compound C, Compound D, Compound E, Compound F, Compound G, Compound H, Compound I, Compound J, Compound K, Compound L, Compound M, and Compound N, or a pharmaceutically-acceptable salt of any of the foregoing, in an amount effective for inhibiting Wip1 phosphatase activity in a cell.

9. The method of claim 8, wherein the compound is Compound L.

10. The method of claim 8, wherein the compound is Compound M.

11. The method of claim 8, wherein the cell is in a host.

12. The method of claim 11, wherein the host is a mammal.

13. The method of claim 12, wherein the mammal is a human.

14. The method of claim 11, wherein the host is afflicted with a disease or condition associated with Wip1 overexpression and the method is effective for treating the disease or condition.

15. The method of claim 14, wherein the disease or condition is cancer.

16. The method of claim 15, wherein the cancer is breast cancer.

17. A pharmaceutical composition comprising a compound selected from the group consisting of Compound A, Compound B, Compound C, Compound D, Compound E, Compound F, Compound G, Compound H, Compound I, Compound J, Compound K, Compound L, Compound M, and Compound N, or a pharmaceutically-acceptable salt of any of the foregoing, and a pharmaceutically-acceptable carrier.

18. The pharmaceutical composition of claim 17, wherein the compound is Compound L.

19. The pharmaceutical composition of claim 17, wherein the compound is Compound M.

20. A method of making a cancer therapeutic composition, which method comprises formulating a compound selected from the group consisting of Compound A, Compound B, Compound C, Compound D, Compound E, Compound F, Compound G, Compound H, Compound I, Compound J, Compound K, Compound L, Compound M, and Compound N, or a pharmaceutically-acceptable salt of any of the foregoing, with a pharmaceutically-acceptable carrier.

21. The method of claim 20, wherein the compound is Compound L.

22. The method of claim 20, wherein the compound is Compound M.

23. A compound selected from the group consisting of Compound A, Compound B, Compound C, Compound D, Compound E, Compound F, Compound G, Compound H, Compound I, Compound J, Compound K, Compound L, Compound M, and Compound N, or a pharmaceutically-acceptable salt of any of the foregoing.

24. A method of screening a compound for Wip1-inhibiting activity, which method comprises comparing the level of Wip1 phosphatase activity in a test sample that has been contacted with the compound to the level of Wip1 phosphatase activity in a control sample that has not been contacted with the compound, wherein a lower level of Wip1 phosphatase activity in the test sample as compared to the control sample is indicative of the ability of the compound to inhibit Wip1.

25. A method of determining the efficacy with which a test compound inhibits Wip1, which method comprises comparing the level of Wip1 phosphatase activity in a test sample that has been contacted with the test compound to the level of Wip1 phosphatase activity in a control sample that has been contacted with a compound that is known to inhibit Wip1, wherein a lower level of Wip1 phosphatase activity in the test sample as compared to the control sample is indicative of the test compound having a greater efficacy for inhibiting Wip1 than the known compound and wherein a higher level of Wip1 phosphatase activity in the test sample as compared to the control sample is indicative of the test compound having a lower efficacy for inhibiting Wip1 than the known compound.
